(19) United States
(12) Patent Application Publication
(10) Pub. No.: US 2026/0260038 A1
(43) Pub. Date: Sep. 3, 2026

(54) SYSTEMS, METHODS, AND INTERFACES FOR PREDICTING COATING WEATHERING

(71) Applicant: PPG Industries Ohio, Inc., Cleveland, OH (US)

(72) Inventors: Daniel Fernando Moyano, Allison Park, PA (US); Sabine Julitta Griesbeck, Frankfurt (DE); Sui Lan Tang, Kuala Lumpur (MY); Om Prakash Choudhary, Pittsburgh, PA (US)

(73) Assignee: PPG Industries Ohio, Inc., Cleveland, OH (US)

(21) Appl. No.: 18/881,800

(22) PCT Filed: Jul. 12, 2023
(86) PCT No.: PCT/US2023/070018

§ 371 Date: Jan. 7, 2025

Related U.S. Application Data
(60) Provisional application No. 63/368,493, filed on Jul. 15, 2022.

(51) Int. Cl. G06F 30/27

(52) U.S. Cl. CPC G06F 30/27

(57) ABSTRACT

A computer-implemented method is disclosed that includes identifying initial color data for an initial coating applied to a target object, wherein the initial color data identifies (i) a coating having a plurality of ingredients, and (ii) colorimetric data for the coating at application; identifying weathered color data for the initial coating on the target object; determining changed colorimetric data between the initial color data and the weathered color data; using the changed colorimetric data to train a machine learning algorithm; receiving an end user selection provided through a graphical user interface of a new coating having a different plurality of ingredients; using the one or more machine learning algorithms to predict a weathered form of the new coating upon application of the plurality of weathering exposure conditions; and displaying data corresponding to the predicted weathered form of the new coating.

### FIELD

The present invention relates to devices, computer-implemented methods, and systems for predicting weathering-based changes to a coating over time.

### BACKGROUND

Modern coatings provide several important functions in industry and society. Coatings can protect a coated material from corrosion, such as rust. Coatings can also provide an aesthetic function by providing a particular color and/or texture to an object. For example, most assets such as automobiles are coated using paints and various other coatings in order to protect the metal body of the automobile from the elements and also to provide aesthetic visual effects.

In view of the wide-ranging uses for different coatings, it is often necessary to identify a target coating composition that can withstand certain weathering, such as to maintain a particular color in a particular locale over a certain amount of time. For instance, it might be necessary to identify a target coating composition that is capable of maintaining a blue metallic hue in a hot and humid locale, while in another case it may be desirable to maintain that color in a cold and dry locale. One or more of the pigments used in such a coating formulation, however, may be more or less stable in various environments. For example, one type of pigment might do particularly well in a humid environment, but may denature more rapidly in cold environments outside of the presence of a stabilizing ingredient.

Some conventional systems attempt to address these problems by monitoring how certain formulations perform over time in given real-use environments. For example, conventional systems may monitor target objects (e.g., automobiles) returned for repainting, and monitoring relative fade or other weathering damage, and maintain such reports for future reference. In other conventional systems, coating manufacturers may attempt to predict how such formulations may perform given the known chemistries of the underlying formulation ingredients. Systems that simply monitor target objects over time may not provide relevant real-time ingredient information for future coatings, which are continually updated over time with different hues and effect pigments that may not have been previously available. Moreover, relying primarily or exclusively on known chemistries of individual components may ignore the protective or degradative effects of other ingredients in the eventual coating, particularly when placed in a given physical environment.

Thus, there are many opportunities for quickly identifying how particular coating formulations may perform in different environments.

### SUMMARY

The present invention provides systems, methods, and computer program products for efficiently and accurately estimating expected weathering of a coating based on the individual and/or combined contributions from individual ingredients. For example, the present invention comprises computerized systems employing methods for identifying real-world weathering of coatings and their constituent ingredients, and determining the individual contributions thereto. The systems can further employ artificial intelligence to predict colorimetric changes from future weather-based degradation using one or more of the previously measured ingredients.

For example, a computer-implemented method for automatically determining and displaying expected weathering after weathering exposure of a coating formula, comprising: identifying initial color data for an initial coating applied to a target object, wherein the initial color data identifies (i) a coating having a plurality of ingredients, and (ii) colorimetric data for the coating at application; identifying weathered color data for the initial coating on the target object, wherein the weathered color data comprises colorimetric data corresponding to the initial coating after the coating applied to the target object has been subject to a plurality of weathering exposure conditions, including at least heat, and light, and water exposure; determining changed colorimetric data between the initial color data and the weathered color data, wherein the changed colorimetric data is associated with (i) each of the coating ingredients, and (ii) the plurality of weathering exposure conditions; processing the changed colorimetric data through one or more machine learning algorithms, wherein the one or more machine learning algorithms store in a database an association of each of the plurality of ingredients with a determined rate of change in colorimetric data upon application of the plurality of weathering exposure conditions; receiving an end user selection provided through a graphical user interface of a new coating having a different plurality of ingredients; using the one or more machine learning algorithms applying the changed colorimetric data, determining, the predicted weathered form of the new coating upon application of the plurality of weathering exposure conditions; and displaying on the graphical user interface data corresponding to the predicted weathered form of the new coating.

In addition, a computerized system can comprise: a database, and one or more computer processors; computer-executable instructions stored on the database, that, when executed, cause the computerized system to perform the following: identify initial color data for an initial coating applied to a target object, wherein the initial color data identifies (i) a coating having a plurality of ingredients, and (ii) colorimetric data for the coating at application; identify weathered color data for the initial coating on the target object, wherein the weathered color data comprises colorimetric data corresponding to the initial coating after the coating applied to the target object has been subject to a plurality of weathering exposure conditions, including at least exposure to heat, light, and precipitation; determine changed colorimetric data between the initial color data and the weathered color data, wherein the changed colorimetric data is associated with (i) each of the coating ingredients, and (ii) the plurality of weathering exposure conditions; and process the changed colorimetric data through one or more machine learning algorithms, wherein the one or more machine learning algorithms store in a database an association of each of the plurality of ingredients with a determined rate of change in colorimetric data upon application of the plurality of weathering exposure conditions.

Additional features and advantages will be set forth in the description which follows, and in part will be obvious from the description, or may be learned by practice. The features and advantages may be realized and obtained by means of the instruments and combinations particularly pointed out in the appended claims and aspects. These and other features will become more fully apparent from the following description and appended claims, or may be learned by the practice of the examples as set forth hereinafter.

### BRIEF DESCRIPTION OF THE DRAWINGS

In order to describe the manner in which the above recited and other advantages and features can be obtained, a more particular description briefly described above will be rendered by reference to specific examples thereof, which are illustrated in the appended drawings. Understanding that these drawings are merely illustrative and are not therefore to be considered to be limiting of its scope, the present invention will be described and explained with additional specificity and detail through the use of the accompanying drawings in which:

FIG. 1A illustrates a schematic overview of a system that is trained with multiple formulas colorimetric measurements in advance of weathering;

FIG. 1B illustrates a schematic overview of system 100 as it continues to receive learning inputs after the panels are subjected to weathering;

FIG. 1C illustrates a schematic overview of a client and server system that can be used to identify individual formula ingredients and each ingredients' individual impact to weathering of the formula;

FIG. 1D illustrates graphs comparing machine learning predictions to actual data after the system has been trained and validated;

FIG. 2 illustrates a schematic in which an end user accesses the system in order to mix and match ingredients and identify in real-time expected weathering data;

FIG. 3 illustrates a method comprising a plurality of acts in a method for automatically determining and displaying expected weathering after weathering exposure of a coating formula; and

FIG. 4 illustrates an additional or alternative method performed by a system configured for automatically determining and displaying expected weathering after weathering exposure of a coating formula.

### DETAILED DESCRIPTION

The present invention provides systems, methods, and computer program products for efficiently and accurately estimating expected weathering of a coating based on the individual and/or combined contributions from individual ingredients. For example, the present invention comprises computerized systems employing methods for identifying real-world weathering of coatings and their constituent ingredients, and determining the individual contributions thereto.

Accordingly, as will be understood more fully herein, the present invention can provide a number of benefits to end users, particularly those looking to optimize not only the appropriate color for refinishing an article, but also for those looking to maintain color consistency over time given local weather patterns and/or durability requirements. Moreover, end users such as asset repair operators and even the end customer can gain confidence that a custom color designed through a graphical user interface will age as expected on the finished product.

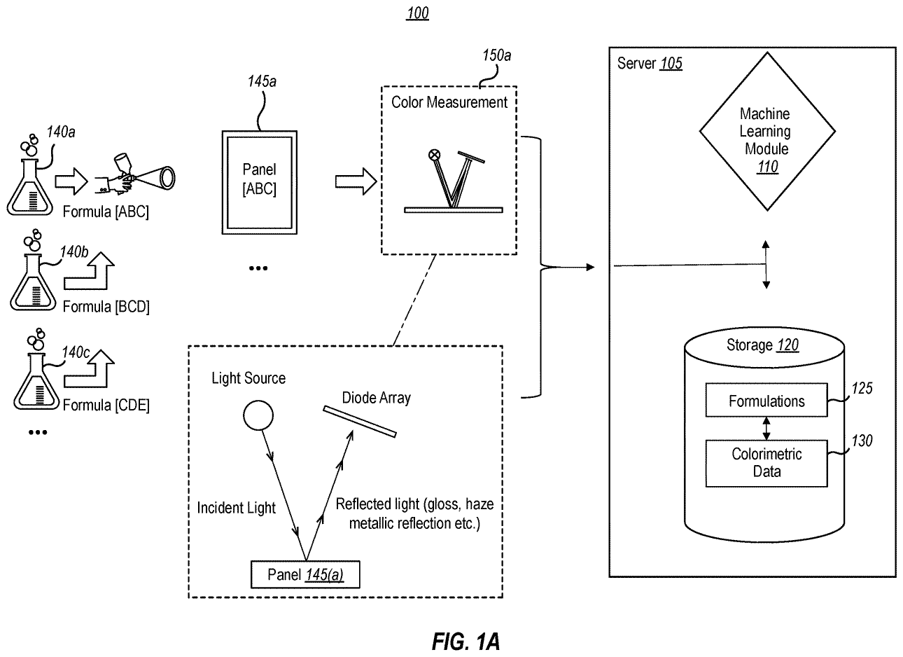

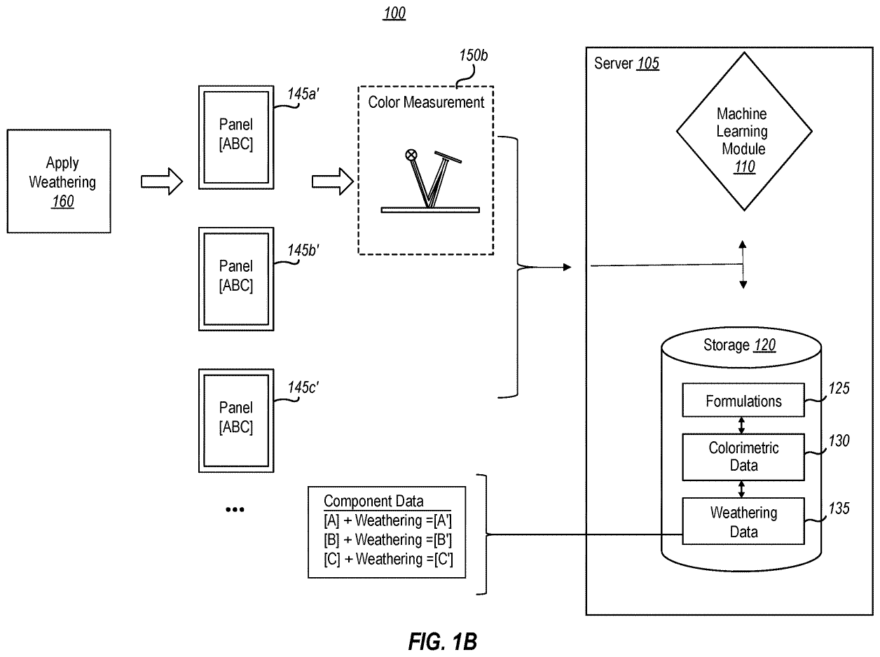

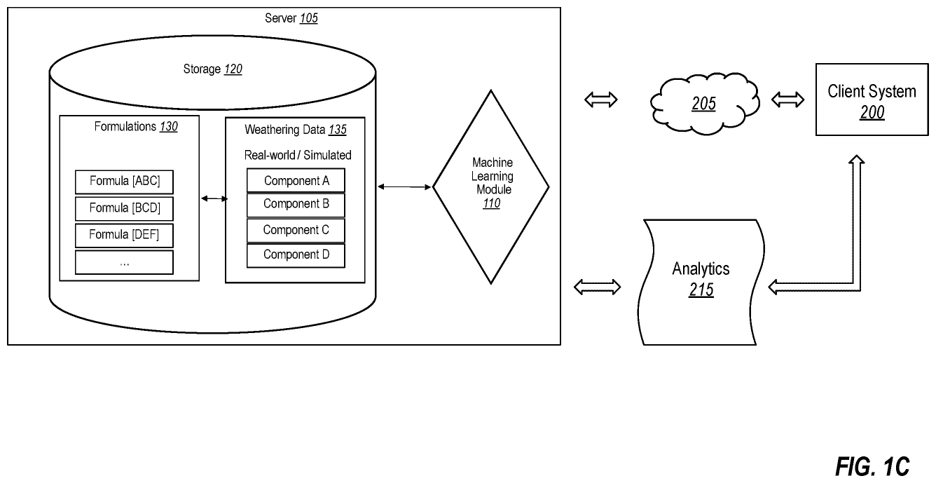

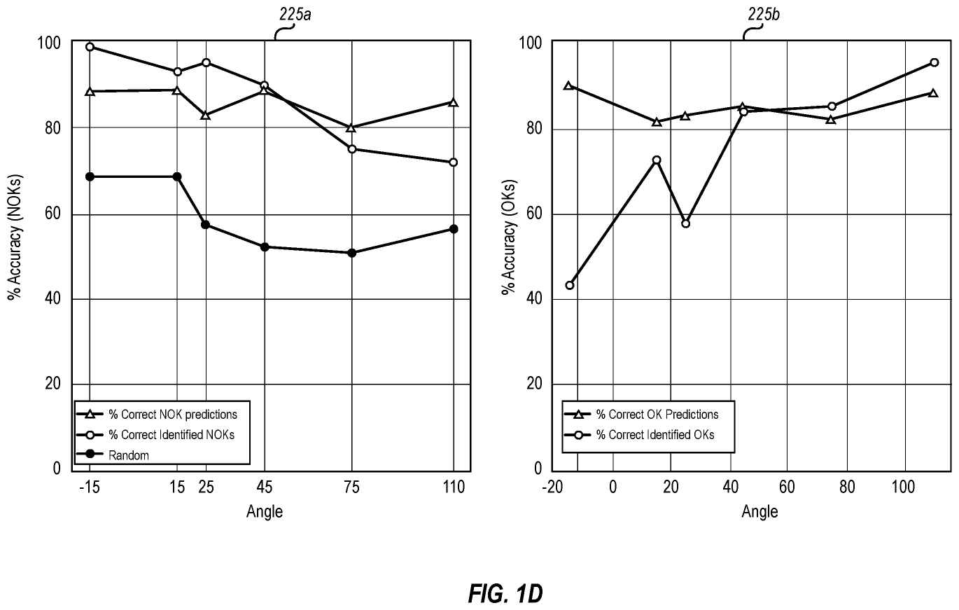

FIG. 1A illustrates a schematic overview of a system that employs one or more machine learning algorithms trained with multiple formulas' colorimetric measurements in advance of weathering. For example, FIG. 1A illustrates that system 100 can comprise a plurality of coating formulas 140a, 140b and 140c, etc., one or more a target object 145 (a, etc.), and a server 105 that is configured to work in the context of input from one or more color measurement steps 150(a).

FIG. 1A further shows that each coating formula 140a, 140b and 140c specifies ingredients and/or subcomponents of a coating. For example, FIG. 1A shows that coating formula 140a includes ingredients “A,” “B,” and “C.” As used herein, the terms “sub-components” and “elements” are used interchangeably with the term ingredients. Moreover, these terms can be understood to mean specific ingredients of a particular name (e.g., a particular formulation of blue pigment at a particular shade), as well as a broad class of such ingredients (pigments or toners of a particular chemical class, e.g., organic/inorganic pigments, binders, solvents, special effect pigments, etc.), or even varying concentrations of the same initial ingredients. Along these lines, FIG. 1A shows that coating formula 140b comprises ingredients “B” “C” and “D,” and coating formula 140c comprises ingredients “C,” “D,” and “E,” and so on. The ingredients may include a base coat and/or one or more colorants toners, pigments, stabilizers, effect pigments, and/or combinations thereof. At least some exemplary ingredients can include pigments such as titanium dioxide, and base coat components such as polyester polyols, as well as solvents such as propyleneglycol methyl ether, and ethyleneglycol hexyl ether.

The operator (or end user) can apply each coating formula 140a, 140b, 140c to the given one or more target objects 145 (e.g., 145a, or others not shown). As illustrated in FIG. 1A, for example, target object 145a is represented by a flat panel. One will appreciate, however, the target object 145a may alternatively comprise a curved or bent metallic sheet. In addition, other target objects 145a of various materials and/or appropriate compositions that best approximate an end-use application are contemplated herein.

FIG. 1A further shows that target object 145a is sprayed at least with formula 140a (i.e., formulation “ABC”). As mentioned, the operator can apply one or more coating formulas directly on target object 145a. For example, although FIG. 1A illustrates one panel 145a with one formula 140a (i.e., formula “ABC”) applied thereto, the operator can also spray other panels (not shown) individually with corresponding formulas 140b and 140c, etc., or may spray the same individual panels with multiple different formulas. In any event, after allowing the given panel(s) to cure, FIG. 1A shows that the operator can perform an initial (i.e., before weathering) color and/or reflectance measurement step of the painted/coated portion(s). As discussed more fully below, the color measurements can be used to train one or more machine learning modules 110 of server 105.

For example, FIG. 1A further illustrates a color measurement step 150a that provides data to server 105. That is, an operator can measure the target object 145 (a) using one or more computerized color or reflectance identification devices, such as a colorimeter, spectrometer, spectrophotometer, and/or an image capture device (e.g., camera). Using the relevant measurement device, the operator can then generate initial color (and/or reflectance) data for an initial coating of each coating formula 140a, 140b, 140c, etc. The operator can then pass the received measurement data for each coating formula 140a, 140b to server 105. Server 105, in turn, performs various learning and storing functions to associate and maintain the measurement data as related to formula ingredients. In one example, server 105 uses the illustrated one or more machine learning modules 110 to coordinate the color measurement 150a data for each sprayed coating formula 140a, 140b, 140c, and further associate each ingredient thereof with the color measurement data. As such, color data, reflectance data, and/or image data, as applicable, obtained via the received measurement step 150a can be understood to comprise “training” sets used to train the one or more machine learning modules 110 for how a formula looks upon application before weathering.

FIG. 1A also shows that server 105 comprises one or more storage devices 120, which in turn comprise various components 125, 130, etc. for storing and relating the formula and color measurement data for use in connection with the one or more machine learning modules 110. For example, FIG. 1A shows that storage 120 comprises various storage components in this case in the form of a formulations component 125 and a colorimetric data component 130. The formulations component 125 may be understood as a data structure that stores formula names, lists of corresponding ingredients, and various properties (or concentrations) thereof before and after weathering. The colorimetric data component 130, by contrast, may be understood as a data structure that holds measurements obtained in the color measurement step 150a, which may include any one or more of image, color, and reflectance data corresponding to the formulas in the formulations component 125.

Such a formal distinction between components and data structures, however, is not necessarily required, and is employed herein primarily for convenience in description. For example, the various storage components 125, 130 may cooperatively store information regarding the ingredients included in each formulation 140 (a-c, etc.), and further associations of images and color/reflectance values for each formulation and ingredient as applied. In either case, and however configured or segmented, the one or more machine learning modules 110 coordinate with storage 120 to learn user associations of formulations, colorimetric data, and related images of the sprayed panels 145.

By way of explanation, the term “module” as used herein means computer executable code that, when executed by one or more processors at a given computer system (e.g., computer system 100, or server 105), causes the given computer system to perform a particular function. By contrast, the term “component,” as used herein, means a passive set of instructions or data structures that store, manage, and/or otherwise provide information handled or otherwise processed through a given module. One of skill in the art, however, will appreciate that the distinction between different modules or components is at least in part arbitrary, and that modules or components may be otherwise combined and/or divided and still remain within the scope of the present disclosure. As such, the description of a given element as being a “module” or a “component” is provided only for the sake of clarity and explanation and should not be interpreted to indicate that any particular structure of computer executable code and/or computer hardware is required, unless expressly stated otherwise. In this description, the terms “component,” “agent,” “manager,” “service,” “engine,” “virtual machine” or the like may also similarly be used.

Referring again to FIG. 1A, the formulations component 125 can comprise current and prior information determined and/or stored about any number of given ingredients (or combinations thereof). Such information includes that formula 140a comprises ingredients A, B, C, while formula 140b comprises ingredients B, C, D, and so on. As previously mentioned, the alternate ingredients B, D, C may comprise adjusted concentrations of original ingredients A, B, C, etc. The formula component further comprises analyses associated with each formula and corresponding ingredient. Such analyses can include associations by machine learning module 110 (or an operator applying training to the module 110) of one or more weathering effects (e.g., FIG. 1B).

The one or more weathering effects can include not just the type of degradation (or “failure type”) associated with each formula and ingredient, but also information about various locales for the weathering, various facility types, and/or information about devices used to recreate these environmental conditions (e.g., weatherometer), which can be used to help identify defects or other forms of degradation. For purposes of this specification and claims, “degradation” means the presence of one or more defects, or defect-causing processes, such as lack of pigment durability, fading, change in gloss, chemical, electrochemical, biological, and physical-based decomposition or decomposition processes, fouling (including biological fouling via growth of biomatter), pitting, rot, rust, delamination, cratering, cracking, or other surface or coating defects and processes promoting the same. Thus, the terms “defect” “degradation” and “failure type” can be understood to be somewhat interchangeable terms in at least some applications herein with respect to weathering effects, particularly when referring to a point in time presence of a defect.

As referred to above, the formulations component 125 can comprise storage of—and/or reference to—a wide range of formulas, ingredients, and other related data. For example, in addition to formulations and measurement data, storage 120 (via formulations component 125 and/or colorimetric data component 130) can store several hundred, thousand, or even millions or more images of sprayed panels 145 corresponding to each formula that represent states prior to, curing, and/or after weathering, as well as the ingredients of each formula in the system. Similarly, as discussed more fully below, any of the data stored can be gathered before, during and after applied weathering (see FIG. 1B). The storage 120 components, 125, 130 further include various human or machine determinations about qualitative issues in the applied formulas. Such determinations can include broad designations of acceptability (e.g., acceptable or “OK,” and unacceptable—Not OK or “NOK”), or similar such determinations made by one of the one or more machine learning processors 110 after sufficient training.

For example, system 100 can employ the one or more learning algorithm modules 110 to learn from various training sets, and improve analysis expertise over time. Such machine learning module 110 algorithms can comprise but are not limited to algorithms understood as supervised learning algorithms, unsupervised learning algorithms, semi-supervised learning algorithms, reinforcement learning algorithms, reinforcement learning algorithms, self-learning algorithms, feature learning algorithms, anomaly detection algorithms, robot learning algorithms, and/or composite versions thereof. In one example, supervised learning algorithms used herein can include (i) a random forest technique, (ii) an XGBoost trees technique, or (iii) a Support Vector Machine technique. Any one or more of the above algorithms can be employed in a deep learning neural network to store, learn, and process images of panels to similar surface features in future images. To improve class imbalance between “NOK” and “OK” classes, analysis methods can employ a combination of an oversampling technique (Synthetic Minority Oversampling Technique, or SMOTE) followed by an undersampling technique (Edited Nearest Neighbors, ENN)—cumulatively called SMOTEENN. In general, SMOTE is an oversampling technique that generates synthetic samples for a minority class in an imbalanced classification dataset. Using ENN after SMOTE can provide an added advantage of removing noise in majority data points.

Table I, below, provides test results showing an improvement in predictions and identifications before and after use of a SMOTEENN technique with a random forest classifier.

TABLE I

| | | | % Correctly | |
|---|---|---|---|---|
| Random Forest | % Correct NOK | % Correct OK | Identified | % Correctly |
| Classifier | Predictions | Predictions | NOKs | Identified OKs |
| Before | 82% | 89% | 97% | 49% |
| SMOTEENN | | | | |
| After | 90% | 75% | 94% | 84% |
| SMOTEENN | | | | |

| | |
|---|---|
| Portion of Correct Predictions | Portion of Correct Identifications |

The following outlines exemplary uses of artificial intelligence and machine learning relative to object and surface identification in accordance with one or more aspects of the present invention. As part of training, and/or eventual processing of data provided during the color measurement step 150a, the one or more machine learning modules 110 can perform image analysis using a “You Only Look Once” (YOLO) style object detection algorithm on images captured by the user. To enable this usage, an operator or user of system 100 first trains the machine learning modules 110 using various types of image segmentation on training images, such as by using object segmentation, instance segmentation, and semantic segmentation to process panels 145 processed before weathering, as well as after weathering. In general, “object segmentation” can be used to characterize images from a generalized point of view, and the system 100 can additionally or alternatively employ “intersection over union” (IOU) in connection with object detection.

By contrast, “instance segmentation” can involve more specifically defining a boundary, or mask, around various weathering defects. For example, as understood with respect to FIG. 1B, the machine learning module 110 in connection with a weathering data component 135 can use instance segmentation to identify specific areas of differential weathering effects on a sprayed panel. “Semantic segmentation,” in turn, involves characterizing each pixel within the boundaries identified in the instance segmentation step. Using semantic segmentation, for example, weathered panels can be mapped by pixel, and associated color value.

As ongoing training commences, the system 100 (i.e., the server 105 and machine learning modules 110) can continue to learn and characterize identified surfaces. For example, the system 100 can employ training on both previously characterized images and new images fed into the system. As previously mentioned, one or more human experts can label segmented images from previously weathered training sets (not shown) to identify surface defects, as well as the types of surface defects, and further provide any other characterizations of the sprayed formulations regarding the same. The expert user can then feed the characterizations for each image back into the neural network of system 100, enabling the system 100 to self-optimize for future application.

Along these lines, a human expert might label a processed image as including various geometric features, such as labels for cone shapes, rectangles, circles, ovals, or the like, and further provide color labels to the extent such labels were not already identified by the system. Other geometric features can include more specific input and corresponding rules, such as that the certain types of fading or discoloration are related to heat factors compared with water exposure, discussed more fully below. Thus, the human expert(s) can train the one or more machine learning modules 110 to understand not just how to identify generalized shapes, but also to understand context about its object identifications that can help ensure future analyses are contextually accurate.

FIG. 1B illustrates a schematic overview of system 100 as it continues to receive learning inputs after the panels 145 (a, b, c, etc.) are subjected to weathering. For example, FIG. 1B illustrates that system 100 further employs weathering in a step 160 on each of the panels (145a, 145b, 145c, etc.). The weathering step 160 can be natural, e.g., by allowing the panel to sit in an outdoor environment for several months or years, or the operator can apply weathering artificially via a weatherometer (not shown). In general, a weatherometer comprises a laboratory scale device into which an operator inserts target objects (e.g., 145 (a, b, c)), and applies simulated weathering phenomena such as UV light, precipitation, cloud coverage, heat index, dew point, and other variations of heat, and “water exposure,” etc. As used herein, “water exposure” means precipitation, and humidity (naturally occurring, or via simulation), as well as applied water, such as water applied through a water spray or jet (e.g., via a weatherometer). A weatherometer can enable an operator to generate weathering effects on a target object 145 over a period of hours, days or weeks rather than a more customary time frame of months or years used to observe natural weathering.

After or even during the weathering step 160, the operator can again gather images and/or color measurement/reflectance data for each panel/target object 145. For example, FIG. 1B shows a second color measurement step 150b, in which the operator/user again measures color and/or reflectance data, such as with a colorimeter, spectrometer, or spectrophotometer. As before with the initial measurement data in step 150a, the operator communicates the second color measurement 150b to the server 105, which includes the machine learning processor 110 and the storage device 120. As discussed more fully below, the operator also provides various indications of acceptability of the color, such as indicating various panels as “OK” or “Not OK” (i.e., “NOK”). Such determinations can include but are not limited to determinations of “delta E” (or “dE”) tolerance levels at different measurement angles, where a delta E of 0 at any given angle is the ideal.

FIG. 1B further shows that storage 120 can comprise one or more additional data structures in the form of weathering data component 135, which can be used to store or otherwise coordinate data measured from step 150b. Again, the specific distinction between components 125, 130, and 135 is somewhat arbitrary, and made for convenience in description. In any event, FIG. 1B shows that the weathered data 135 includes at least colorimetric data for each ingredient of the coating formula 140a, 140b, 140c and any other weathering data for each ingredient, such as OK/NOK, specific points of degradation. The weathered data for the formula ingredients in component 135 are represented for ingredient “[A]” as “[A′].” Similarly, measured data after weathering for ingredient “[B]” is shown as “[B′],” weathered data for ingredient “[C]” is shown as “[C′],” and so on. As mentioned, in at least one case, the difference between “[A]” and “[A′]” can be expressed in terms of delta E colorimetric differences. The differences illustrated as [A′], [B′], and [C′] can correspond to evaluations based on images showing various defects, such as pigment durability, change in gloss, delamination, rust, or other forms of degradation, and any measurements (color, reflectance, etc.) that can be correlated to those effects.

For example, as mentioned above, the operator can provide qualitative assessments for each weathered panel 145a′, 145b′, and 145c′. The qualitative assessments can include broad characterizations such as “okay,” or “not okay” (i.e., “NOK”) based on a particular tolerance of delta E, or observations regarding types of fading, change in gloss, color intensity, delamination, or other defects observed. The qualitative assessments can further include specific identifications of degradation on the panel, such as point by human identifications of fading, bubbling, rust, or still other form of degradation observed. The operator's qualitative assessments for each panel 145a′, 145b′, 145c′, etc. can be passed through the one or more machine learning modules 110 and stored with other data in the weathering data component 135. To the extent the determinations of OK and NOK result in an imbalanced dataset, the server 105 can further employ SMOTEENN to oversample the minority dataset. These techniques, in sum, enable the one or more machine learning modules 110 to also learn to associate specific changes over the entire dataset in image data, colorimetric (e.g., delta E), and/or reflectance data across the surface of any given weathered panel 145 (a′-c′, etc.) with both qualitative and numerical assessments, and thus to help ferret out contributions of specific ingredients.

The resulting determinations by the one or more machine learning modules 110 can include determinations of rates of change in gloss, fading, or other appearance metrics, including constitution of the coating on the object in one form or another due to weathering. For example, the one or more machine learning modules 110 may determine that ingredient or element C of formula 140a has a tendency to fade, induce a change in gloss, or delaminate at a higher or lower rate than another ingredient or element in the presence of humidity, or in the presence of UV light, or some combination thereof with other weathering factors. Similarly, the one or more machine learning modules 110 may ascertain that ingredient B of formula 140b appears to have a stabilizing effect on element C of the same formula, such that the combination of ingredient B with C tends to cause formulations including ingredients B and C together to withstand degradation in the same type of environment, or perhaps accelerate degradation in another type of environment. The same analysis could apply simply by varying the concentration of ingredient B, or the concentrations of B and/or C.

FIG. 1C illustrates a schematic overview of a client and server system that can be used to identify individual formula ingredients and each ingredients' correlation or contribution to weathering data. For example, FIG. 1C illustrates that server 105 is in communication with one or more client system 200 over network 205. Network 205 can comprise any local, wide, regional, or global area network connection, ranging anywhere from a direct wire connection to a connection over the Internet. Network 205 can also or additionally comprise local near-field networks, including Bluetooth and WIFI networks. In any event, FIG. 1C shows that client system 200 interacts with server 105 data to assist with training the one or more machine learning modules, evaluate the progress thereof, and to obtain predictions corresponding to certain formula ingredients and their response to the weathering step 160.

For example, FIG. 1C shows that client system 200 communicates with server 105, such as via network 205. The client system 200 can comprise an end user terminal of several different types, including but not limited to a traditional personal computing device, or a mobile device such as a laptop, tablet, mobile phone, or other form of network connected computing device. A network connected client system, however, is not required, and an operator may interface directly with server 105. In other words, the server 105 and database 120 may be construed as a standalone or other database application installed on client system 200. In any event, the operator can interact with server 105 via multiple different means in order to enter data, train the one or more machine learning modules 110 with pre- and post-weathering measurements (e.g., analytics 215). Such operator interaction (e.g., via the one or more analytics messages 215) can further include monitoring the progress of the one or more machine learning modules 110 in terms of the ability of the one or more machine learning modules 110 to provide consistent or accurate predictions, and/or adjustments to the predictions (e.g., after application of SMOTEENN techniques).

Along these lines, FIG. 1D provides various example results charts (225a, 225b) showing exemplary progress of machine learning (in this case using a Random Forest classifier) predictions in light of various training sets provided by the operator to server 105. The charts of FIG. 1D plot the percentage of correctly identified “OKs” (225a) and “NOKs” (225b) versus angles of measurement (e.g., steps 150a, 150b) using a “delta E” measurement for all angles. As shown, the one or more machine learning modules 110 can generate analyses and predictions that perform better than a random baseline (i.e., the lower line of chart 225a) using a realistic, supervised class distribution.

For example, charts 225a and 225b show the results of test data used to verify how well the machine learning algorithms outlined herein can perform in real-world scenarios using weathered data. The data presented in the charts describe both the recall and precision metrics traditionally used to assess the accuracy of computer predictions. For example, chart 225a shows that the percentage “Not OKs” (NOKs) at each measured angle, where “NOK predictions” refers to the ratio of NOK predictions (computer-generated data using the one or more machine learning algorithms) that were correct out of all NOK computer-generated predictions (i.e., precision), while “Identified NOKs” refers to the amount actual NOK real samples that the model was able to correctly label as NOK (i.e., recall). Along these lines, chart 225a shows that computer predictions exceeded accuracy of a random control. Moreover, chart 225a shows that the lines showing percent of correct NOK identifications are roughly of similar shape and magnitude to the line showing % correct NOK identifications, indicating the overall high power of prediction/accuracy of the models.

By contrast, chart 225b compares the % correctly identified OKs (OK recall) and % correct OK predictions, i.e., using computer-generated predictions based on the one or more machine learning algorithms (OK precision). As with the NOK measurements in chart 225a, the OK measurements and predictions in chart 225b show that the computer predictions performed well for the majority of the angles, and only fails in the low angles where variability from the measurement creates noise on the models. In other words, the charts show that the use by the one or more machine learning algorithms of delta E data to predict weathering degradation can closely approximate real-world observations. That is, the fact that the both computer prediction precision and recall metrics for both OK and NOK are high (generally over 75% threshold for accuracy) confirms that the computer-assisted models and algorithms outlined herein can correctly differentiate OK and NOK characteristics in most cases, thus confirming validity of the ingredient-specific approaches used to generate the predictions.

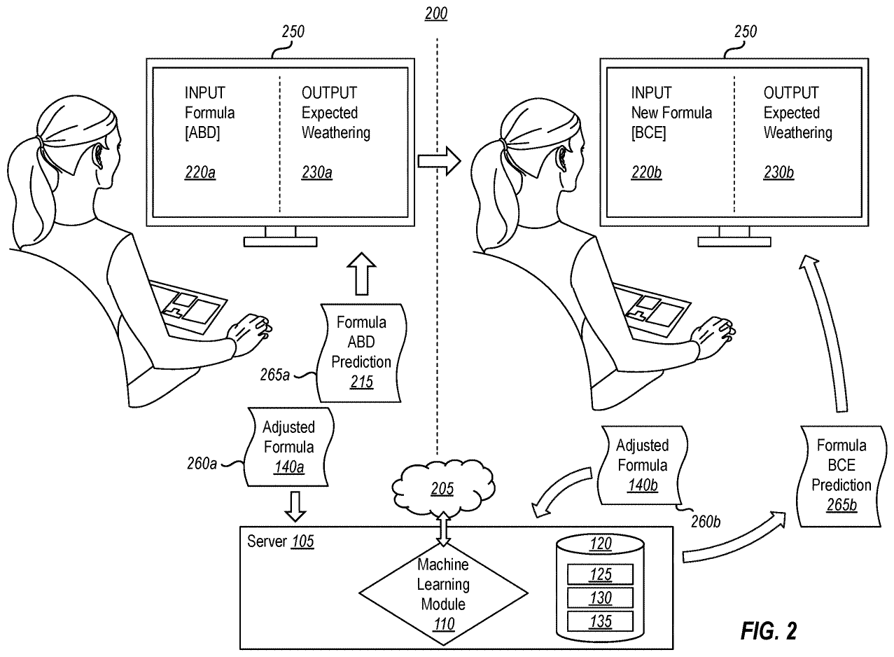

FIG. 2 illustrates a schematic in which an end user uses client system 200 in order to mix and match ingredients and identify in real-time expected weathering data for a new formulation. For example, FIG. 2 illustrates an overview of a client system 200 comprising graphical user interface 250 in various sequences of input and output interfaces (220a/220b, and 230a/230b, respectively). Through a first input interface 220, the user inputs a set of ingredients based for example on formula 140a, albeit substituting ingredient C for ingredient D or varying concentrations thereof. For example, ingredient D may be an alternate concentration of ingredient C. The computer system 200 sends the new formula to server 105 via one or more messages 20a. For example, the user may be interested in using a formula of a particular color and new effect pigment (element D, such as “mica”), whereas only system 100 only has prior measurements for the color with a different effect pigment (e.g., element C, such as “aluminum”), and the client 200 submits the input to server 105 over network 205. The one or more machine learning modules 110 at server 105 can process data from the one or more messages 260a. In one example, the one or more machine learning algorithms 110 extrapolate stored weathering data associated with each ingredient to ascertain their respective contributions to degradation in one or more other/new formulations (e.g., measured from formula 140b and 140c).

That is, the one or more machine learning modules 110 can then prepare a predicted weathering effect of combining the data from measurements pertaining to formula 140a with contributions of element D from pre/post measurements of formulas 140b and 140c (and potentially others). Thus, in response to an input request in interface 220a, the user interface 250 may provide an output 230a (sent via one or more messages 260b) that indicates that in response to a particular type of weathering, the new formula ABD is predicted to succeed, improve, or fail by some amount, or some rate of change, relative to the original baseline formula 140a. For example, the output interface 230a may indicate that the requested formula ABD will fade, change in adhesion or gloss, or degrade by 5% within 9 years compared to 11 years for the original formula, or otherwise degrade by 20% in different type of accelerated testing (e.g. different weatherometer conditions). Alternatively, the output interface 230a can indicate that the input formula of ABD will last longer (e.g., 12 years) on one or more of those same factors than an expected duration (11) without such defects by changing one of the ingredients to a new formula, e.g., changing formula ABD to ABE.

Along these lines in a next illustrated example, the user similarly inputs another new formula BCE in input interface 220b, where new formula BCE is a variation on the original formula 140b (i.e., varying formula BCD with ingredient E). The computer system 200 sends the user's input to the server 105 via one or more input messages 260b, indicating that the input is an adjustment of formula 140b by inserting ingredient E for original ingredient D. FIG. 2 shows then that the server 105 generates one or more predictions for the altered formula, and returns one or more weathering predictions 265b. The computer system 200 then displays the prediction information in the output screen 230b.

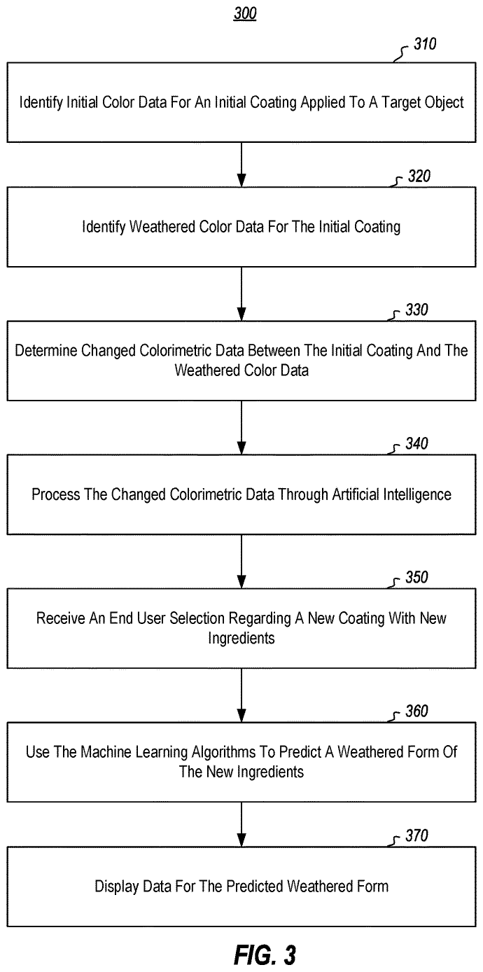

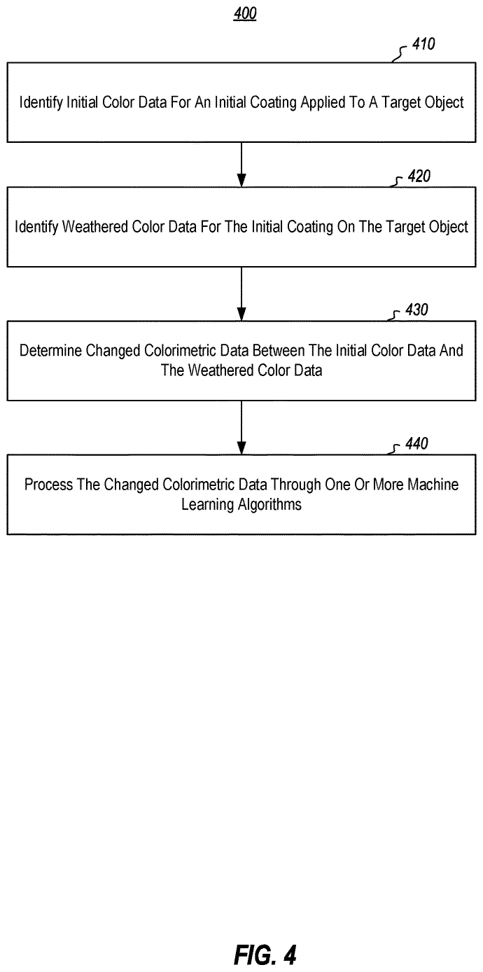

Accordingly, FIGS. 1 through 2 provide multiple components, modules and schematics as part of a system for automatically determining and predicting expected weathering effects of various individual ingredients of a coating/composition. The present invention can also be described in terms of one or more methods for accomplishing a particular result. Along these lines, FIGS. 3 and 4 illustrate various methods for automatically determining and displaying expected weathering after weathering exposure of a coating formula. The acts and steps of FIGS. 3 and 4 are discussed below with reference to the systems components and modules of FIGS. 1A-2.

For example, FIG. 3 illustrates that a method 300 of automatically determining and displaying expected weathering after weathering exposure of a coating formula can include an act 310 of identifying initial color data for an initial coating applied to a target object. Act 310 includes identifying initial color data for an initial coating applied to a target object, wherein the initial color data identifies (i) a coating having a plurality of ingredients, and (ii) colorimetric data for the coating at application. For example, FIG. 1A shows that various formulations, their corresponding ingredients, and their initial color measurement (via step 150a) as applied to a target object 145(a) can be passed to and stored in server 105 via storage 120.

FIG. 3 also shows that method 300 can comprise an act 320 of identifying weathered color data for the initial coating. Act 320 includes identifying weathered color data for the initial coating on the target object, wherein the weathered color data comprises colorimetric data corresponding to the initial coating after the coating applied to the target object has been subject to a plurality of weathering exposure conditions, including at least heat, light, and water exposure. For example, FIG. 1B shows that the operator performs a second color measurement 150b on the various target objects after the applied weathering step 160. As previously described, weathering exposure conditions can include naturally and synthetically applied (e.g., via a weatherometer) weather in the form of heat, light, and water exposure in the form of precipitation, and humidity, as well as water applied through a water spray or jet, and so forth.

In addition, FIG. 3 shows that method 300 can comprise an act 330 of determining changed colorimetric data between the initial coating and the weathered color data. Act 330 includes determining changed colorimetric data between the initial color data and the weathered color data, wherein the changed colorimetric data is associated with (i) each of the coating ingredients, and (ii) the plurality of weathering exposure conditions. For example, FIG. 1B shows that server 105 can identify ingredient-specific associations (e.g., stored in component 135) with various weathering (applied, or natural) at least in part by comparing color measurements taken in steps 150a (initial measurements) and 150b (measurements after weathering).

Furthermore, FIG. 3 shows that method 300 can comprise an act 340 of processing the changed colorimetric data through artificial intelligence. Act 340 can comprise processing the changed colorimetric data through one or more machine learning algorithms, wherein the one or more machine learning algorithms store in a database an association of each of the plurality of ingredients with a determined rate of change in colorimetric data upon application of the plurality of weathering exposure conditions. For example, FIGS. 1A-1B show that the one or more machine learning algorithms process pre- and post-weathering data in correlation with various formulations, and store the associations in storage 120. As part of the processing, the one or more machine learning algorithms can identify various associations between individual ingredients and the identified changes in color measurements (e.g., delta E or “dE” between steps 150a and 150b).

Still further, FIG. 3 shows that method 300 can include an act 350 of receiving an end user selection regarding a new coating with new ingredients. Act 350 can include receiving an end user selection provided through a graphical user interface of a new coating having a different plurality of ingredients. For example, FIG. 2 shows that the user provides input through input interface 220a of an adjustment to formula 140a, such as by swapping an ingredient C in original formula 140a with ingredient D.

FIG. 3 also shows that method 300 can include an act 360 of using the machine learning algorithms to predict a weathered form of the new ingredients. Act 360 can include using the one or more machine learning algorithms applying the changed colorimetric data, determining, the predicted weathered form of the new coating upon application of the plurality of weathering exposure conditions. For example, FIG. 2 shows that, in response to the user input requesting predicted weathering of the adjusted formula 140a (i.e., message 260a), the server 105 sends a response via message 265a, which includes weathering predictions for the requested, adjusted formula. Message 265a can include, for example, delta E predictions made by the one or more machine learning modules 110 for coating formulas taken at various angles, and given certain assumptions about expected weathering.

In addition, FIG. 3 shows that method 300 can include an act 370 of displaying data for the predicted weathered form. Act 370 can include displaying on the graphical user interface a data corresponding to the predicted weathered form of the new coating. For example, FIG. 2 shows that the input and output interfaces (220a/b, 230a/b, respectively) can be provided, and can be displayed on display device 250 of the client computer system 200. As previously discussed, the predicted weathered form for the new coating can include predicted changes to the new coating based on the new coating's plurality of ingredients and a plurality of weathering exposure conditions. The predicted changes may include changes in color, adhesion, gloss and/or other appearance metrics, such as fouling.

The predicted weathered form for the new coating may also include a prediction about whether the new coating will fail (e.g., a change in gloss or adhesion beyond a predetermined value) after exposure to weathering exposure conditions for a certain amount of time. The predicted weathered form for the new coating may also include a prediction about what type of failure the new coating will experience; for example, a change in gloss and/or adhesion. The predictions can further include variations in expected weathering based on particular climate conditions in a particular geographic location. For example, the response from server 205 can include that the new formulation will degrade outside an acceptable tolerance within 10 years in a cold, dry climate, while degrading outside an acceptable tolerance within 8 years in a wet, hot climate.

FIG. 4 illustrates that an additional or alternative method 400 for automatically determining and displaying expected weathering after weathering exposure of a coating formula can include an act 410 of identifying initial color data for an initial coating applied to a target object. Act 410 includes identify initial color data for an initial coating applied to a target object, wherein the initial color data identifies (i) a coating having a plurality of ingredients, and (ii) colorimetric data for the coating at application. For example, FIG. 1A shows that an operator applies one or more formulas comprising a plurality of ingredients to a panel, and then performs a color measurement step 150a to identify at least an initial colorimetric measurement (i.e., pre-weathering) for the applied formulation.

FIG. 4 also illustrates that method 400 can comprise an act 420 of identifying weathered color data for the initial coating. Act 420 includes identifying weathered color data for the initial coating on the target object, wherein the weathered color data comprises colorimetric data corresponding to the initial coating after the coating applied to the target object has been subject to a plurality of weathering exposure conditions, including at least exposure to heat, light, and precipitation. For example, FIG. 1B shows application of a weathering step 160 (i.e., naturally or via a weatherometer). The weathering step can include application of light, heat, water, humidity, and other forms in various combinations outlined herein. The operator can then perform a second or subsequent color measurement step 150b to identify at least any differences in colorimetric data due to the applied weathering.

In addition, FIG. 4 illustrates that method 400 can comprise an act 430 of determining changed colorimetric data between the initial coating and the weathered color data. Act 430 includes determining changed colorimetric data between the initial color data and the weathered color data, wherein the changed colorimetric data is associated with (i) each of the coating ingredients, and (ii) the plurality of weathering exposure conditions. For example, FIG. 1B shows that a second color measurement step 150b is performed after the applied weathering step 160. The operator can pass the second color measurement data for the given panel to server 105, where the color measurements can be tied to the various formulations.

Furthermore, FIG. 4 illustrates that method 400 can comprise an act 440 of processing the changed colorimetric data through artificial intelligence. Act 440 includes processing the changed colorimetric data through one or more machine learning algorithms, wherein the one or more machine learning algorithms store in a database an association of each of the plurality of ingredients with a determined rate of change in colorimetric data upon application of the plurality of weathering exposure conditions. For example, server 105 comprises one or more machine learning modules 110, which apply any number of one or more supervised learning algorithms, such as a random forest technique, an XGBoost trees technique, and a Support Vector Machine technique. The one or more machine learning modules 110 can, as previously discussed, determine various contributions of individual ingredients of a formula by analysis of one or more formulas containing that ingredient, and how the various formulas responded to the weathering step 160. The one or more machine learning modules 110 can process the change in color measurements from step 150b relative to step 150a, and determine changes in colorimetric data, reflectance, or the like as applied to each individual ingredient of each formula.

Though not illustrated, the computer-implemented methods 300 and 400 may further comprise recommending one or more ingredient substitutions. The one or more ingredient substitutions may be optimized by the one or more machine learning modules 110 to minimize changes in appearance in response to the plurality of weathering exposure conditions. That is, the one or more ingredient substitutions may be optimized by the one or more machine learning algorithms employed by the modules 110 to resist the predicted changes due to the plurality of weathering exposure conditions.

Accordingly, FIGS. 1A-4 provide a number of systems, components, and modules that can be used to significantly increase both speed and accuracy of contributions by individual ingredients to weathering responses. Part of this increase can be provided by use of varying types of weathering, including employing both natural and synthesized weathering conditions. Part of the increase can also be due to use of one or more machine learning algorithms to process and associate changes in instrument-measured data, and associate those changes with individual ingredients. Thus, the principles outlined in this disclosure can provide end users with significantly more efficient and accurate tools to get instant feedback about weathering degradation implications on new formulas with new ingredients.

The present invention may comprise or utilize a special-purpose or general-purpose computer system that includes computer hardware, such as, for example, one or more processors and system memory, as discussed in greater detail below. The scope of the present invention also includes physical and other computer-readable media for carrying or storing computer-executable instructions and/or data structures. Such computer-readable media can be any available media that can be accessed by a general-purpose or special-purpose computer system. Computer-readable media that store computer-executable instructions and/or data structures are computer storage media. Computer-readable media that carry computer-executable instructions and/or data structures are transmission media. Thus, by way of example, and not limitation, the invention can comprise at least two distinctly different kinds of computer-readable media: computer storage media and transmission media.

Computer storage media are physical storage media that store computer-executable instructions and/or data structures. Physical storage media include computer hardware, such as RAM, ROM, EEPROM, solid state drives (“SSDs”), flash memory, phase-change memory (“PCM”), optical disk storage, magnetic disk storage or other magnetic storage devices, or any other hardware storage device(s) which can be used to store program code in the form of computer-executable instructions or data structures, which can be accessed and executed by a general-purpose or special-purpose computer system to implement the disclosed functionality of the invention.

Transmission media can include a network and/or data links which can be used to carry program code in the form of computer-executable instructions or data structures, and which can be accessed by a general-purpose or special-purpose computer system. A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer system, the computer system may view the connection as transmission media. Combinations of the above should also be included within the scope of computer-readable media.

Further, upon reaching various computer system components, program code in the form of computer-executable instructions or data structures can be transferred automatically from transmission media to computer storage media (or vice versa). For example, computer-executable instructions or data structures received over a network or data link can be buffered in RAM within a network interface module (e.g., a “NIC”), and then eventually transferred to computer system RAM and/or to less volatile computer storage media at a computer system. Thus, it should be understood that computer storage media can be included in computer system components that also (or even primarily) utilize transmission media.

Computer-executable instructions comprise, for example, instructions and data which, when executed at one or more processors, cause a general-purpose computer system, special-purpose computer system, or special-purpose processing device to perform a certain function or group of functions. Computer-executable instructions may be, for example, binaries, intermediate format instructions such as assembly language, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The invention may also be practiced in distributed system environments where local and remote computer systems, which are linked (either by hardwired data links, wireless data links, or by a combination of hardwired and wireless data links) through a network, both perform tasks. As such, in a distributed system environment, a computer system may include a plurality of constituent computer systems. In a distributed system environment, program modules may be located in both local and remote memory storage devices.

Those skilled in the art will also appreciate that the invention may be practiced in a cloud-computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources (e.g., networks, servers, storage, applications, and services). The definition of “cloud computing” is not limited to any of the other numerous advantages that can be obtained from such a model when properly deployed.

A cloud-computing model can be composed of various characteristics, such as on-demand self-service, broad network access, resource pooling, rapid elasticity, measured service, and so forth. A cloud-computing model may also come in the form of various service models such as, for example, Software as a Service (“SaaS”), Platform as a Service (“PaaS”), and Infrastructure as a Service (“IaaS”). The cloud-computing model may also be deployed using different deployment models such as private cloud, community cloud, public cloud, hybrid cloud, and so forth.

A cloud-computing environment, or cloud-computing platform, may comprise a system that includes one or more hosts that are each capable of running one or more virtual machines. During operation, virtual machines emulate an operational computing system, supporting an operating system and perhaps one or more other applications as well. Each host may include a hypervisor that emulates virtual resources for the virtual machines using physical resources that are abstracted from view of the virtual machines. The hypervisor also provides proper isolation between the virtual machines. Thus, from the perspective of any given virtual machine, the hypervisor provides the illusion that the virtual machine is interfacing with a physical resource, even though the virtual machine only interfaces with the appearance (e.g., a virtual resource) of a physical resource. Examples of physical resources include processing capacity, memory, disk space, network bandwidth, media drives, and so forth.

In view of the foregoing, the present invention may be embodied in multiple different configurations, as outlined above, and as exemplified by the following.

For example, one configuration can include a computer-implemented method for automatically determining and displaying expected weathering after weathering exposure of a coating formula, comprising: identifying initial color data for an initial coating applied to a target object, wherein the initial color data identifies (i) a coating having a plurality of ingredients, and (ii) colorimetric data for the coating at application; identifying weathered color data for the initial coating on the target object, wherein the weathered color data comprises colorimetric data corresponding to the initial coating after the coating applied to the target object has been subject to a plurality of weathering exposure conditions, including at least heat, light, and water exposure; determining changed colorimetric data between the initial color data and the weathered color data, wherein the changed colorimetric data is associated with (i) each of the coating ingredients, and (ii) the plurality of weathering exposure conditions; processing the changed colorimetric data through one or more machine learning algorithms, wherein the one or more machine learning algorithms store in a database an association of each of the plurality of ingredients with a determined rate of change in colorimetric data upon application of the plurality of weathering exposure conditions; receiving an end user selection provided through a graphical user interface of a new coating having a different plurality of ingredients; using the one or more machine learning algorithms applying the changed colorimetric data, determining, the predicted weathered form of the new coating upon application of the plurality of weathering exposure conditions; and displaying on the graphical user interface data corresponding to the predicted weathered form of the new coating.

In an additional or alternative configuration, the weathering conditions comprise subjecting the target object to natural weathering in a natural environment over a multi-month period. The computer-implemented method as recited in any of the preceding claims, wherein the weathering conditions comprise a simulation of weathering conditions through a weatherometer. In an additional or alternative configuration, the method can further include identifying a historical dataset for the initial coating; wherein the historical data set includes the changed colorimetric data associated with the plurality of weathering exposure conditions, and one or more different applications of the initial coating on a different target object that was subjected to a different plurality of weathering exposure conditions. In an additional or alternative configuration, the method can further include using the one or more machine learning algorithms applying the historical data set to determine an expected change in colorimetric data for each of the ingredients associated with each weathering condition in the plurality of weathering exposure conditions and in the different plurality of weathering exposure conditions. In an additional or alternative configuration, the method can further include determining a failure type for the initial coating after application of the plurality of weathering exposure conditions; wherein the failure type comprises change in color.

In an additional or alternative configuration, the method can further include determining a failure type for the initial coating after application of the plurality of weathering exposure conditions; wherein the failure type comprises change in gloss, or a change in adhesion. In an additional or alternative configuration, the method can further include receiving a user selection reflecting one or more changes in ingredients of the new coating; determining a change in weathering of the new coating upon application of any of the plurality of weathering exposure conditions. In an additional or alternative configuration, the method can further include displaying on the graphical interface predicted changes to the new coating after a plurality of time intervals that simulate application of the plurality of weathering exposure conditions.

In an additional or alternative configuration, the predicted changes to the new coating show changes in adhesion. In an additional or alternative configuration, the predicted changes to the new coating show changes in coloration. In an additional or alternative configuration, the one or more machine learning algorithms employ a supervised learning algorithm to compute the expected weathering of the new coating formula. In an additional or alternative configuration, the supervised learning algorithm comprises one or more of (i) a random forest technique, (ii) an XGBoost trees technique, or (iii) a Support Vector Machine technique. In an additional or alternative configuration, the method can further include displaying predicted colorimetric values for the selected coating formula; and displaying one or more changes in predicted colorimetric values for the selected coating formula after one or more future time intervals.

In an additional or alternative configuration, the present disclosure may provide a computerized system having a database, and one or more computer processors; computer-executable instructions stored on the database, that, when executed, cause the computerized system to perform the following: identify initial color data for an initial coating applied to a target object, wherein the initial color data identifies (i) a coating having a plurality of ingredients, and (ii) colorimetric data for the coating at application; identify weathered color data for the initial coating on the target object, wherein the weathered color data comprises colorimetric data corresponding to the initial coating after the coating applied to the target object has been subject to a plurality of weathering exposure conditions, including an exposure to heat, light, and precipitation; determine changed colorimetric data between the initial color data and the weathered color data, wherein the changed colorimetric data is associated with (i) each of the coating ingredients, and (ii) the plurality of weathering exposure conditions; and process the changed colorimetric data through one or more machine learning algorithms, wherein the one or more machine learning algorithms store in a database an association of each of the plurality of ingredients with a determined rate of change in colorimetric data upon application of the plurality of weathering exposure conditions.

In an additional or alternative configuration, the weathered color data is measured via at least a spectrophotometer. In an additional or alternative configuration, the weathered color data represents a plurality of measurements of the initial coating on the target object over a multi-year time frame. In an additional or alternative configuration, the weathered color data represents a plurality of measurements of the initial coating on the target object over a multi-month time frame. In an additional or alternative configuration, the weathered color data represents a plurality of measurements of colorimetric data for the initial coating at multiple different angles of measurement. In an additional or alternative configuration, the system is further configured to: receive an end user selection provided through a graphical user interface of a new coating having a different plurality of ingredients, wherein the different plurality of ingredients differs by any the base coat or any of the one or more ingredients in the initial coating.

In an additional or alternative configuration, the system is further configured to: use the one or more machine learning algorithms applying the changed colorimetric data to determine a predicted weathered form of the new coating upon application of the plurality of weathering exposure conditions; and display on the graphical user interface data corresponding to the predicted weathered form of the new coating. In an additional or alternative configuration, the system is further configured to: display one or more ingredient substitution recommendations for the new coating; wherein the one or more ingredient substitution recommendations are optimized by the one or more machine learning algorithms to minimize change in appearance in response to the plurality of weather conditions. In an additional or alternative configuration, the displayed one or more ingredient substitution recommendations comprise a change in concentration of any ingredient of the plurality of ingredients.

Although the subject matter has been described in language specific to structural features and/or methodological acts, and whereas particular examples of this disclosure have been described above for purposes of illustration, it will be evident to those skilled in the art that numerous variations of the details of the present disclosure may be made without departing from what is defined in the appended claims. In particular, it is to be understood that the subject matter defined in the appended claims is not necessarily limited to the described features or acts described above, or the order of the acts described above. Rather, the described features and acts are disclosed as example forms of implementing the claims.

1-23. (canceled)

24. A computer-implemented method for automatically determining and displaying expected weathering after weathering exposure of a coating formula, comprising:
identifying initial color data for an initial coating applied to a target object, wherein the initial color data identifies (i) a coating having a plurality of ingredients, and (ii) colorimetric data for the coating at application;
identifying weathered color data for the initial coating on the target object, wherein the weathered color data comprises colorimetric data corresponding to the initial coating after the coating applied to the target object has been subject to a plurality of weathering exposure conditions, including at least heat, light, and water exposure;
determining changed colorimetric data between the initial color data and the weathered color data, wherein the changed colorimetric data is associated with (i) each of the coating ingredients, and (ii) the plurality of weathering exposure conditions;
processing the changed colorimetric data through one or more machine learning algorithms, wherein the one or more machine learning algorithms store in a database an association of each of the plurality of ingredients with a determined rate of change in colorimetric data upon application of the plurality of weathering exposure conditions;
receiving an end user selection provided through a graphical user interface of a new coating having a different plurality of ingredients;
using the one or more machine learning algorithms applying the changed colorimetric data, determining, the predicted weathered form of the new coating upon application of the plurality of weathering exposure conditions; and
displaying on the graphical user interface data corresponding to the predicted weathered form of the new coating.

25. The computer-implemented method as recited in claim 24, wherein the weathering conditions comprise subjecting the target object to natural weathering in a natural environment over a multi-month period.

26. The computer-implemented method as recited in claim 24, wherein the weathering conditions comprise a simulation of weathering conditions through a weatherometer.

27. The computer-implemented method as recited in claim 24, further comprising:
identifying a historical dataset for the initial coating;
wherein the historical data set includes the changed colorimetric data associated with the plurality of weathering exposure conditions, and one or more different applications of the initial coating on a different target object that was subjected to a different plurality of weathering exposure conditions.

28. The computer-implemented method as recited in claim 27, further comprising:
using the one or more machine learning algorithms applying the historical data set to determine an expected change in colorimetric data for each of the ingredients associated with each weathering condition in the plurality of weathering exposure conditions and in the different plurality of weathering exposure conditions.

29. The computer-implemented method as recited in claim 24, further comprising:
determining a failure type for the initial coating after application of the plurality of weathering exposure conditions;
wherein the failure type comprises change in color.

30. The computer-implemented method as recited in claim 24, further comprising:
determining a failure type for the initial coating after application of the plurality of weathering exposure conditions;
wherein the failure type comprises change in gloss, or a change in adhesion.

31. The computer-implemented method as recited in claim 24, further comprising:
receiving a user selection reflecting one or more changes in ingredients of the new coating;
determining a change in weathering of the new coating upon application of any of the plurality of weathering exposure conditions.

32. The computer-implemented method as recited in claim 31, further comprising:
displaying on the graphical interface predicted changes to the new coating after a plurality of time intervals that simulate application of the plurality of weathering exposure conditions.

33. The computer-implemented method as recited in claim 31, wherein:
the predicted changes to the new coating show changes in adhesion.

34. The computer-implemented method as recited in claim 31, wherein
the predicted changes to the new coating show changes in coloration.

35. The computer-implemented method as recited in claim 24, wherein:
the one or more machine learning algorithms employ a supervised learning algorithm to compute the expected weathering of the new coating formula.

36. The computer-implemented method as recited in claim 35, wherein the supervised learning algorithm comprises one or more of (i) a random forest technique, (ii) an XGBoost trees technique, or (iii) a Support Vector Machine technique.

37. The computer-implemented method as recited in claim 24, further comprising:
displaying predicted colorimetric values for the selected coating formula; and
displaying one or more changes in predicted colorimetric values for the selected coating formula after one or more future time intervals.

38. A computerized system comprising:
a database, and one or more computer processors;
computer-executable instructions stored on the database, that, when executed, cause the computerized system to perform the following:
identify initial color data for an initial coating applied to a target object, wherein the initial color data identifies (i) a coating having a plurality of ingredients, and (ii) colorimetric data for the coating at application;
identify weathered color data for the initial coating on the target object, wherein the weathered color data comprises colorimetric data corresponding to the initial coating after the coating applied to the target object has been subject to a plurality of weathering exposure conditions, including an exposure to heat, light, and precipitation;
determine changed colorimetric data between the initial color data and the weathered color data, wherein the changed colorimetric data is associated with (i) each of the coating ingredients, and (ii) the plurality of weathering exposure conditions; and
process the changed colorimetric data through one or more machine learning algorithms, wherein the one or more machine learning algorithms store in a database an association of each of the plurality of ingredients with a determined rate of change in colorimetric data upon application of the plurality of weathering exposure conditions.

39. The computerized system of claim 38, wherein:
the weathered color data is measured via at least a spectrophotometer.

40. The computerized system of claim 38, wherein:
the weathered color data represents a plurality of measurements of the initial coating on the target object over a multi-year time frame.

41. The computerized system of claim 38, wherein:
the weathered color data represents a plurality of measurements of the initial coating on the target object over a multi-month time frame.

42. The computerized system of claim 38, wherein:
the weathered color data represents a plurality of measurements of colorimetric data for the initial coating at multiple different angles of measurement.

43. The computerized system as recited in claim 42, wherein the system is further configured to:
receive an end user selection provided through a graphical user interface of a new coating having a different plurality of ingredients, wherein the different plurality of ingredients differs by any the base coat or any of the one or more ingredients in the initial coating.

44. The computerized system as recited in claim 43, wherein the system is further configured to:
use the one or more machine learning algorithms applying the changed colorimetric data to determine a predicted weathered form of the new coating upon application of the plurality of weathering exposure conditions; and
display on the graphical user interface data corresponding to the predicted weathered form of the new coating.

45. The computerized system as recited in claim 43, wherein the system is further configured to:
display one or more ingredient substitution recommendations for the new coating;
wherein the one or more ingredient substitution recommendations are optimized by the one or more machine learning algorithms to minimize change in appearance in response to the plurality of weather conditions.

46. The computerized system as recited in claim 45, wherein the displayed one or more ingredient substitution recommendations comprise a change in concentration of any ingredient of the plurality of ingredients.
